(19) United States
(12) Patent Application Publication
(10) Pub. No.: US 2019/0150734 A1
(43) Pub. Date: May 23, 2019

(54) CONTACT LENS FOR OBSERVING AND TREATING AN EYE WITH AN INCIDENT LIGHT BEAM

(71) Applicant: PHAKOS, Montreuil (FR)

(72) Inventor: Olivier AUMAITRE, Montreuil (FR)

(21) Appl. No.: 16/098,755

(22) PCT Filed: May 6, 2016
(86) PCT No.: PCT/FR2016/051074

§ 371 Date: Nov. 2, 2018

(51) Int. Cl. A61B 3/125; A61B 3/117; A61N 5/06

(52) U.S. Cl. CPC A61B 3/125; A61N 2005/0648; A61N 5/06; A61B 3/117

(57) ABSTRACT

A contact lens for examining an eye under an incident light beam includes a main element having: a planar first entrance face placed perpendicularly to the beam; at least one reflective face; and a spherical exit face having a centre of curvature and an axis of symmetry that is perpendicular to the entrance face and that passes through the centre of curvature, the exit face being intended to be applied to the eye and to focus the beam on a working point in the interior of the eye, a second entrance face placed facing the spherical exit face, along the axis of symmetry; and a central void extending from the planar first entrance face to the second entrance face.

### CROSS-REFERENCE TO RELATED APPLICATIONS

This patent application is a U.S. nationalization under 35 U.S.C. § 371 of International Application No. PCT/FR2016/051074, filed May 6, 2016, the disclosure of which is incorporated herein by reference in its entirety.

### FIELD OF THE DISCLOSURE

The present disclosure relates generally to a contact lens for examination of an eye under an incident light beam. It finds application in particular for the observation of the interior of the eye and/or its treatment by irradiation, mainly at the retina, so as to detect various disorders affecting among others the pupil, the crystalline lens or the retina, which necessitate being inspected optically in an accurate manner.

The present disclosure also relates to a method of obtaining such a contact lens.

### BACKGROUND

Various contact lenses for the observation and treatment of an eye by an incident light beam are known, such as the Goldmann lens. Conventionally, the contact lens for the observation and the treatment of an eye by an incident light beam comprises a main element comprising a planar entrance face, a spherical exit face to be applied to the cornea and a reflective face. This type of lens can also comprise a plurality of other reflective faces distributed over the circumference of the lens. This is a one-piece axially symmetric body with an axis of symmetry aa′. In the operating condition, this axis aa′ passes through the retina of a subject at a point F. To reach a point Fx corresponding to a point near the axis aa′ of the eye, a light beam X, with axis xx′, must enter into this lens (commonly called a Goldmann lens) perpendicular to the entrance face, at a certain distance from the axis aa′, and pass directly, without reflection, through the lens and the eye. To reach a second point Fy corresponding to a point situated far from the axis aa′, a light beam Y must, after its entrance into the lens perpendicular to the entrance face, undergo total reflection by the reflective face at a point in order to penetrate into the eye at an incidence angle i. To each region of the retina distant from the axis aa′ corresponds a particular inclination of the reflective face with respect to the planar entrance face.

Such contact lenses are however costly to manufacture due to the volume of matter composing the main element and the method which must be implemented to obtain a contact lens having the expected optical qualities to allow the observation and the treatment of an eye using an incident beam. In fact, contact lenses of the prior art define main elements of which the volume is delimited axially by the first entrance face and the exit face and, radially, by the reflective face (and possibly several other reflective faces) as well as portions of the circumference of the main element. These one-piece contact lenses thus comprise a volume filled with material.

With a view to ensuring a satisfactory eye observation and treatment quality by the incident light beam, it is indispensable that the contact lens allow, due to its shape and composition qualities, satisfactory focusing of the beam and the retention of its focalization qualities for any point of the eye, mainly of the interior chamber of the eye. It has been noted that one-piece contact lenses of this kind comprising such a volume filled with material necessitate particular precautions when obtaining them so as to preserve the expected optical properties for allowing the observation and the treatment of an eye by an incident beam. In fact, the method for obtaining such contact lenses require design effort in order to avoid the phenomenon of material shrinkage, such as for example the determination of the thicknesses of the different portions of the contact lens as well as the determination of the temperatures to which these must be cooled. A contact lens including a volume thus filled with material therefore further requires a relatively long cooling step which negatively impacts the cost of obtaining such a contact lens.

Furthermore, the methods for obtaining contact lenses according to the prior art require a metallization step on the faces of the part requiring optical qualities, such as the first entrance face, the reflective face(s) and the spherical exit face. A metallization step of this type is costly and prolongs the time for obtaining a contact lens.

Consequently, in order to limit costs, the contact lenses of the prior art are designed to be re-used numerous times. This imposes that these lenses are capable of enduring sterilization conditions in a hospital environment. According to international standards, sterilization is obtained by remaining in an autoclave brought to 125° C. for 10 minutes. The corresponding French standard is even more constraining because it requires a stay at 134° C. for 20 minutes. These sterilization procedures require frequent and costly sterilization operations.

This background information is included to provide some information believed by the applicant to be of possible relevance to the present disclosure. No admission is intended, nor is such an admission to be inferred or construed, that any of the preceding information constitutes prior art against the present disclosure.

### SUMMARY

In this context, the problem posed here is to mitigate the aforementioned disadvantages. In particular, the disclosed embodiments relate to a contact lens for the observation and treatment of an eye by a light beam which is intended for a single use, is reliable and the manufacture of which is inexpensive.

The disclosed embodiments also relate to a contact lens that is lightweight, ergonomic, sterile and safe. To this end and according to a first aspect of the present disclosure, a solution proposed by the present disclosure is a contact lens for the examination and/or the treatment of an eye under an incident light beam, comprising a main element having:

a planar first entrance face, disposed perpendicular to the beam,

at least one reflective face, and

a spherical exit face having a center of curvature and an axis of symmetry that is perpendicular to the entrance face and passing through the center of curvature, the exit face being designed to be applied to the eye and to focus the beam on a working point in the interior of the eye.

The main element comprises:

a second entrance face disposed facing the spherical exit face, along the axis of symmetry, and

a central void extending from the planar first entrance face until the second entrance face.

In one embodiment, the second entrance face defines the bottom of the void, the second entrance face being configured to allow the observation of a point in proximity to an axis of symmetry of the eye. The planar first entrance face can also be configured to allow the observation of a point distant from the axis of symmetry of the eye.

In one embodiment, the first entrance face comprises an observation zone, the second entrance face is connected to the first entrance face by an internal lateral wall of the void, and an optical prism is in particular delimited by the observation zone, the reflective face and the internal lateral wall of the void, the prism being configured to allow the penetration of the light beam through the observation zone before undergoing reflection by the reflective face at one point so as to penetrate into the eye at an incidence angle.

In one embodiment, the prism is further delimited by two lateral facets extending from the circumference of the lens to join the reflective face.

In one embodiment, the lens comprises a plurality of distinct reflective faces and a corresponding number of distinct prisms.

In one embodiment, the lens comprises connecting faces defining the circumference of the lens, which are interleaved between the prisms so as to connect the prisms together.

In one embodiment, each connecting face is delimited by two lateral facets of two respective prisms.

In one embodiment, the lens comprises a homogeneous and constant thickness defined between the internal lateral wall and the connecting face that faces it.

In one embodiment, the first entrance face has an opening communicating with the central void.

In one embodiment:

the exit face is delimited by a circular contour delimiting the exit face of a base of the lens,

the second face is delimited by a contour,

the image of the virtual projection of the contour of the exit face, along the axis of symmetry, is contained in the interior of the contour of the second face.

In one embodiment, the image of the virtual projection of the contour of the exit face is a circle inscribed in the contour of the second face.

In one embodiment, the contour of the second face has a polygonal shape, such as hexagonal.

In one embodiment, a contour of the opening has the same geometric shape as the contour of the second entrance face.

In one embodiment, the peaks of the contour of the opening and the peaks of the contour of the second entrance face join to form the edges in the interior of the void, which delimit the internal lateral walls.

In one embodiment:

the contour of the opening has identical dimensions to the contour of the second entrance face, or,

the contour of the opening has greater dimensions than the dimensions of the contour of the second entrance face so that the area defined by the contour of the opening is greater than the area defined by the second entrance face (this feature can for example be observed by projecting these contours on the same plane).

In one embodiment, the lens is one-piece and consists of a transparent plastic material.

According to a second aspect of the present disclosure, a method for obtaining a contact lens according to any one of the embodiments presented in the description is also encompassed.

In one embodiment, the contact lens is obtained by injecting a plastic material into the interior of a mold.

### BRIEF DESCRIPTION OF THE DRAWINGS

The disclosed embodiments will be better understood upon reading the description that follows and upon examining the drawings which accompany it. These drawings are given only by way of illustration and in no way limit the disclosure:

FIG. 1 illustrates a section view along a longitudinal plane of symmetry of the contact lens according to one exemplary embodiment and a schematic representation of the eye on the cornea of which the contact lens is applied,

FIG. 2 shows a top view of the contact lens according to one exemplary embodiment,

FIGS. 3a-3b illustrate section views along the longitudinal symmetry plane of the contact lens according to an exemplary embodiment; FIGS. 3a, 3b and 3c illustrate respectively views along section planes A-A, B-B and C-C which are shown in FIG. 2,

FIG. 4 shows a side view of one exemplary embodiment,

FIG. 5 illustrates a perspective view from above of an exemplary embodiment of the contact lens,

FIG. 6 illustrates a perspective view from below of an exemplary embodiment of the contact lens,

FIG. 7 shows a perspective view of an exemplary embodiment of the contact lens.

### DETAILED DESCRIPTION OF EMBODIMENTS

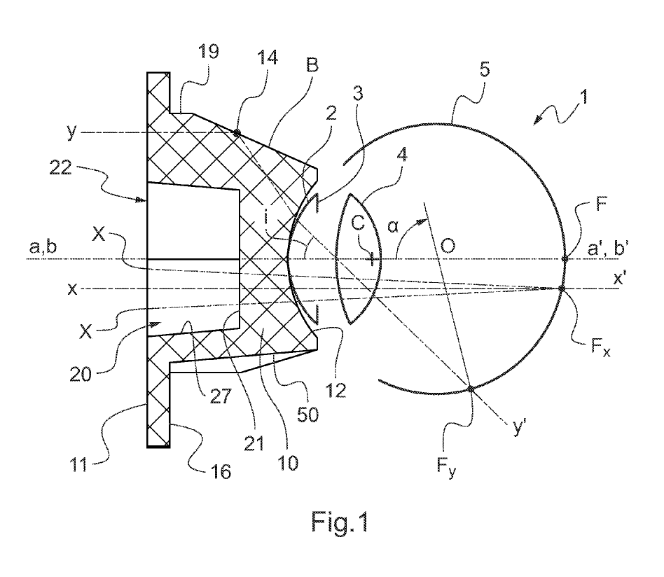

A schematic representation of the eye 1 is shown in FIG. 1. It is an axisymmetric body having an axis of symmetry aa′ which comprises, going from the exterior to the interior of the eye, a cornea 2 by which the light beams enter, an iris 3 the opening of which controls the quantity of material, a crystalline lens 4 and a retina 5, with a spherical shape having a center 0, which the axis aa′ passes through at a point F.

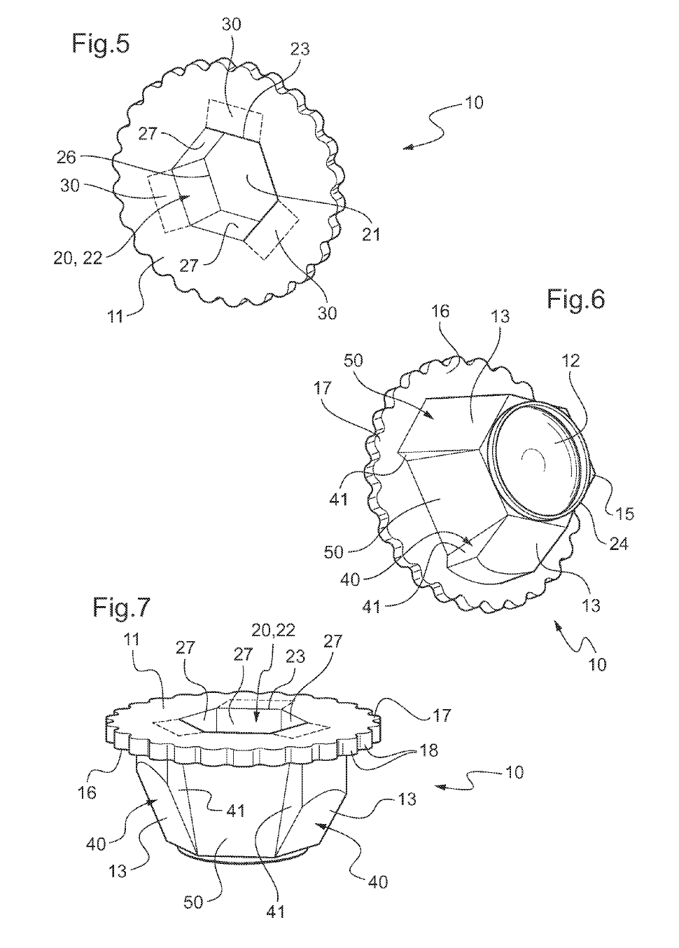

In FIGS. 1 to 7 is illustrated a contact lens for the observation and the treatment of an eye by an incident light beam F. This lens 10 comprises a substantially planar first entrance face 11, an exit face 12 applied to the cornea 2, and a reflective face 13 (reflecting toward the interior of the lens 10). The reflective face 13 allows the indirect observation of the anterior chamber of the eye by reflection of the beams on this wall, and, in some embodiments, by total reflection of the beams on this wall. As can be seen in FIGS. 6 and 7, other reflective faces 13 can further be distributed around the circumference of the lens. The angle between a reflective face 13 and the entrance face 11 depends on the target point on the retina. In some embodiments, this axis can vary from about 50° to about 80°. The perpendicular straight line to the entrance face 11 and passing through the center of curvature C of the exit face 12 defines the axis of symmetry bb′ of the contact lens 30. The axes aa′ and bb′ are conflated here. The dimensions of the lens allow it to tilt on the cornea so as to be able to displace, within certain limits, the observation or treatment point, also called the working point, in the interior of the eye. The axis bb′ then pivots around the center C.

FIG. 1 shows the progression of light in the contact lens according to the present disclosure for two typical point of the retina. The first point, denoted Fx in FIG. 1, corresponds to a point in proximity to the axis aa′ of the eye. To reach this point, the incident light beam X, with axis xx′, enters into the lens 10 perpendicular to the entrance face 11, at a certain distance from the axis aa′, and pass through directly, without reflection, the lens 10 and the eye 1. The second point, denoted Fy, corresponds to a point situated far from the axis aa′. The angle formed by the axis aa′ and the straight line connecting the point F to the center 0 of the retina 5 can exceed 90°. To reach the point F, a light beam Y, of which only the axis yy′ is shown, after its entrance into the lens 10 perpendicular to the face 11, undergoes reflection by the face 13 at a point 14 so as to penetrate into the eye 1 at an angle of incidence i. To each region of the retina 5 distant from the axis aa′, corresponds a particular inclination of the face 13 with respect to the face 11 of the lens 10. That is why the same lens according to the present disclosure can advantageously comprise several reflective faces with different inclinations.

As can be seen in FIGS. 1-5 and 7, the first entrance face 11 is a planar surface extending substantially perpendicularly with respect to the straight line passing through the center of curvature C of the exit face 12 defining the exit axis bb′ of the contact lens 10.

The one-piece lens 10 consists of a material transparent to useful radiation substantially delimited by a shape having a longitudinal symmetry plane. The lens consists of a shape the base 15 of which is substantially hexagonal, developing from the exit face 12 until the planar first entrance face 11 so that the cross section of the circumference of the lens in proximity to the first entrance face 11 is relatively greater than the cross section of the lens in proximity to the exit face 12. The first entrance face 11 is connected to the circumference of the lens at the location of a third connecting face 16 arranged substantially parallel to the first face 11. A skirt 17 having a longitudinal symmetry plane and consisting of an axially symmetric cylindrical volume is thus defined between the first entrance face 11 and the third connecting face 16. In some embodiments, this skirt 17 comprises on its circumference protrusions 18 extending radially toward the exterior and allowing easier gripping of the lens 10 during manipulation of the lens by the operator.

The first entrance face 11 has at its center an opening 22 which communicates with a central void or recess 20 extending from the first entrance face 11 until a second entrance face 21 disposed facing the spherical exit face 12 along the axis of symmetry bb′. Advantageously, the central void 20 is configured to allow the observation and/or the treatment of a point near the axis of symmetry aa′ of the eye via the second entrance face 21. In other words, the central void 20 is configured to allow, during the observation and/or the treatment of a point near the axis of symmetry aa′, the optical progression of the light beam through the central void 21 before passing directly through the second entrance face 21 and the spherical exit face 12 to attain the targeted point. The shape of the void or recess 21 is thus configured to allow the direct visualization, without reflection and via a progression of the beam through the void, of the entire exit face 12 and in particular of its contour 26. In some embodiments, following a section plane passing through the first entrance face 11, the opening 22 is delimited in its interior by a contour 23 of substantially polygonal shape, the number of sides of the polygon being adaptable according to the number of reflective faces 13 included in the main element on its circumference. According to the embodiment shown in FIGS. 1-7, the contact lens includes three reflective faces 13, the opening 22 has a contour 23 of substantially hexagonal shape.

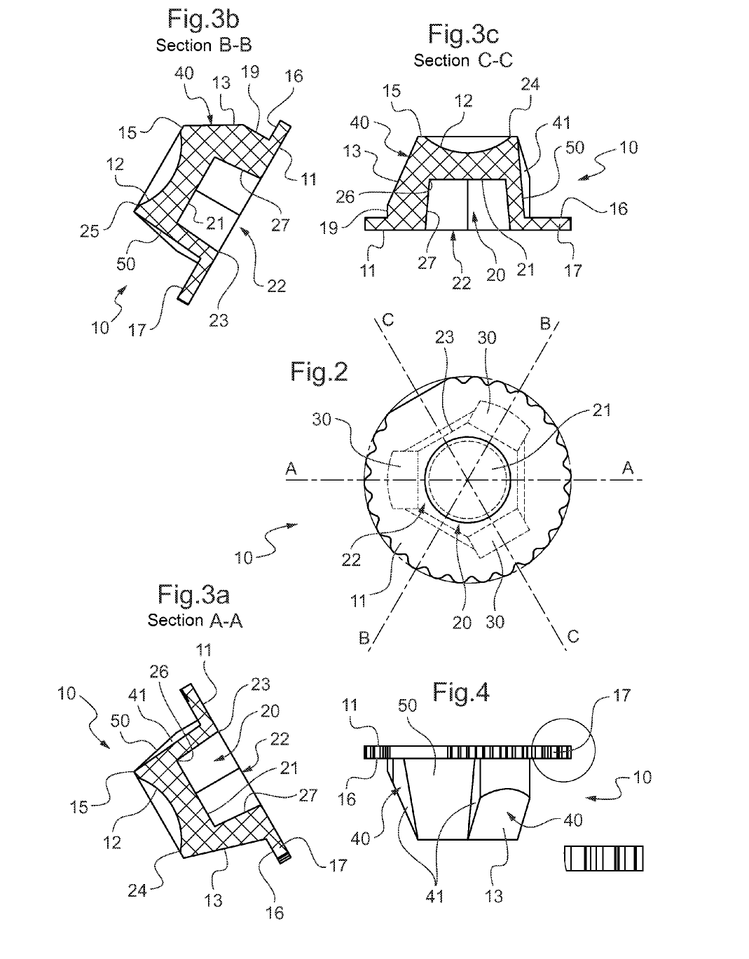

The spherical exit face 12 is delimited by a contour 24 defining a circle, which delimits a spherical exit face 12 of the base 15 of the contact lens. No embodiment in which the shape of the hexagonal base is replaced by another polygonal or circular shape departs from the scope of the present disclosure. As indicated in the description, when the contour 24 delimiting the spherical exit face 12 describes a circle the center of which is a point of intersection between the axis of symmetry of the spherical exit face 12 passing through the curvature point C and a plane extending along the base 15 of the lens, the radius R of this circle can be adapted so that a circle having as its center this point of intersection and having a radius R is inscribed in the shape of the second entrance face 21 and more precisely into its contour 26. Of course, the inscription of this circle necessitates projecting virtually the image of the circle into the contour 26 of the second entrance face 21, or the reverse. Similarly, when the contour 24 delimiting the spherical exit face 12 describes a circle having as its center the intersection point and having radius R, the radius R of this circle can be adapted so that it is inscribed in the contour 23 of the opening 22 of the first entrance face 11. Analogously, the inscription of this circle necessitates projecting virtually the image of the circle into the contour 23 of the first entrance face 11, or the reverse. With reference to FIG. 2, the void 21 is configured to allow the visualization of the entire contour 24 of the exit face 12 through the opening 22.

The second entrance face 21, defining the bottom of the central void 20, is connected to the first entrance face 11 by one or more internal lateral walls 27. More particularly, the internal lateral walls 27 each extend from the edge(s) of the contour 26 of the second entrance face 21 until the corresponding edges of the contour 23 of the opening of the first entrance face 11. In one embodiment, the contour 23 of the opening 22 of the first entrance face has a geometric shape identical to that of the contour 26 of the second entrance face 21 and has identical dimensions. In this case, the internal lateral walls 27 are rectangular; these lateral walls can, according to another example, be trapezoidal. In another embodiment, the contour 23 of the opening 22 of the first entrance face 11 has a geometric shape identical to that of the contour 26 of the second entrance face 21, and has dimensions relatively greater than the dimensions of the contour 26 of the second entrance face 21 so that the area defined by the contour 23 of the opening 22 of the first entrance face 11 is greater than the area defined by the second entrance face 21. This advantageously allows facilitating the de-molding of the contact lens.

As regards the progression of the light, it will be different depending on what the operator desires to observe. When the operator desires to observe the point Fx corresponding to a point near the axis aa′ of the eye, the incident light beam X passes through the central void 20, then pass directly through, without reflection, the second entrance face 21, the spherical exit face 12, then the eye of the subject. When the operator desires to observe the point Fy corresponding to a point distant from the axis aa′ of the eye, the light beam Y, after its entrance into the lens 10 perpendicular to the face 11 via an observation zone 30, undergoes reflection on the reflective face 13 at a point 14 in order to penetrate into the eye at an incidence angle i. The number of observation zones 30 on the first entrance face 11 is adapted according to the number of reflective faces 13 that the contact lens 10 comprises. In other words, the operator can directly see through the second entrance face 21 if he desires to observe a point near the axis aa′ of the eye, or to look through the observation zone 30 if he desires to observe a point distant from the axis aa′ of the eye.

The spherical exit face 12 constitutes, for the contact lens, a concave surface. For the selection of a radius of curvature of the exit face 12, a value greater than the radius of curvatures of the anterior face of the cornea of the eye at rest can be used in order to allow obtaining good contact between the lens and the cornea.

The reflective face 13 is a planar surface extending from the base 15 of the lens and developing toward the third connecting face 16. This reflective face can be connected directly to the third connecting face 16 (FIG. 3a) or be connected via a connecting portion 19 connecting the reflective face 13 to the third connecting face (FIGS. 3b and 3c). To each region of the retina 5 distant from the axis aa′, corresponds a particular inclination of the face 13 with respect to the face 11 of the lens 10. Thus, a plurality of reflective faces 13 each having a different inclination allows maximizing the coverage of the regions of the retina which can be observed.

With reference to the progression of the light in the case of a point distant from the axis aa′ of the eye, as indicated earlier in the description, the first entrance face 11 comprises an observation zone 30 by which the light beam Y penetrates. Once the beam Y penetrates through the observation zone 30 it enters into a block of material 40 having optical properties such as a prism 40. This prism 40 is delimited by the observation zone 30, the reflective face 13 and the internal lateral wall 27 of the central void 20 which is disposed facing the corresponding reflective face 13. It can also be delimited by the connecting portion 19 connecting the reflective face 13 of the third connecting face 16. In FIGS. 1 to 7 is shown an embodiment in which the prisms 40 are delimited by two lateral facets 41 extending from the circumference of the lens to join the reflective face 13, the circumference (corresponding to the periphery of the lens 10 where there is not reflective face 13), the third connecting face 16 and the base 15 (and the connecting portions 19 if there are any). The optical properties of this prism 40 thus allow observing via the observation zone 30 a point distant from the axis of aa′ of the eye via the reflective face 19. Thus delimited, the prism allows the contact lens to be made lighter. Alternatively, when the prism 40 is connected by means of a connecting portion 19, this can be conflated with the facets 41 and form a surface of revolution connecting the reflective face 13, the third connecting face 19, the circumference of the lens and the base 15.

As illustrated, the observation zone 30 can be delimited by a substantially rectangular shape. It is delimited by the internal lateral wall 27 and by the shape of the prism 40. In other words, the observation zone 30 corresponds to a portion of the surface of the first face 11 which is associated with a side of the prism 40. The observation zone 30 has a contour at the interior of which the observation of a point distant from the axis aa′ is possible, while the directly adjacent zones of the first face 11 do not allow such a point to be observed.

According to the embodiment in which there is a plurality of reflective faces 13, there is a corresponding number of distinct prisms 40. What is meant here by distinct is the fact that each prism 40 is associated with a single observation zone 30 and with a single reflective face 13 which belong to it, which signifies that observing in a first observation zone 30 of a first prism 40 allows observing the image returned by the first reflective face 13. On the other hand, observing in the first observation zone 30 associated with the first prism 40, distinct from the second prism 40, does not allow observing the image returned by a second reflective face 13 associated with the second prism 40.

In one embodiment, the contact lens 10 comprises at least two distinct prisms 40. In FIGS. 1 to 7 is shown an embodiment in which the contact lens 10 comprises three distinct prisms 40.

As can be seen in FIGS. 6 and 7, the connecting faces 50 are interleaved between the prisms 40 and connect the prisms 40 to the circumference of the lens 10. Typically, one connecting face 50 is delimited by two lateral facets 41 of two respective prisms, the third connecting face 16 and the exterior edge of the base 15. Alternatively, when the connecting portion 19 is conflated with the facets 41 so as to form a single surface of revolution connecting the reflective face 13, the third connecting face 19, the circumference of the lens and the base 15, the connecting face 50 is delimited by this connecting surface of revolution, the third connecting face 19 and the exterior edge of the base. In one embodiment, a homogenous and constant thickness is defined between the internal lateral wall 27 and the connecting face 50 which faces it. In some embodiments, the thickness is less than 6 mm. In some embodiments, the thickness is less than 3 mm. In some embodiments, the thickness is less than 2 mm. This makes it possible to lighten the contact lens. This also makes it possible to balance the material composing the lens and to avoid material shrinkage which can occur in a large volume of material. These connecting faces 50 have no optical function and the progression of a light beam in a volume of material defined between the connecting face 50 and the internal lateral wall 27 does not allow a subject's fundus to be observed.

In illustrative embodiments, the material used for the production of the lens is a material which does not undergo shrinkage after thermal loading, such as polymethyl methacrylate for example.

According to a second object of the present disclosure, a method for obtaining a contact lens 10 according to any one of the embodiment described above is also covered. In one embodiment, the contact lens is obtained by an injection molding method. The method therefore comprises in particular the injection of material into the interior of a mold configured to form a contact lens 10 according to any one of the aforementioned embodiments so that the lens 10 is formed from a single molded part. In particular, the mold is configured to form a void 20 in the lens 10, the void extending toward the interior of the lens 10 from the planar first entrance face 11 until the second entrance face 21.

1. A contact lens for the examination and/or the treatment of an eye under an incident light beam, the contact lens comprising a main element comprising:
a planar first entrance face, disposed perpendicular to the beam,
at least one reflective face,
a spherical exit face having a center of curvature and an axis of symmetry that is perpendicular to the first entrance face and passing through the center of curvature, the exit face configured to be applied to the eye and to focus the beam on a working point in an interior of the eye,
a second entrance face disposed facing the spherical exit face, along the axis of symmetry, and
a central void extending from the planar first entrance face until the second entrance face.

2. The contact lens according to claim 1, wherein:
the second entrance face defines the bottom of the void, the second entrance face being configured to allow the observation of a point in proximity to an axis of symmetry of the eye, and
the planar first entrance face is configured to allow the observation of a point distant from the axis of symmetry of the eye.

3. The contact lens according to claim 2, wherein:
the first entrance face comprises an observation zone,
the second entrance face is connected to the first entrance face by an internal lateral wall of the void, and
an optical prism is delimited by the observation zone, the reflective face and the internal lateral wall of the void, the prism being configured to allow the penetration of the light beam through the observation zone before undergoing total reflection by the reflective face at a point so as to penetrate into the eye at an incidence angle.

4. The contact lens according to claim 3, wherein the prism is further delimited by two lateral facets extending from a circumference of the lens to join the reflective face.

5. The contact lens according to claim 4, wherein the lens further includes a plurality of distinct reflective faces and a corresponding number of distinct prisms.

6. The contact lens according to claim 5, wherein the lens further includes connecting faces defining the circumference of the lens, which are interleaved between the prisms so as to connect the prisms together.

7. The contact lens according to claim 6, wherein each connecting face is delimited by two lateral facets of two respective prisms.

8. The contact lens according to claim 6, wherein the lens further includes a homogeneous and constant thickness defined between the internal lateral wall and the connecting face that faces it.

9. The contact lens according to claim 1, wherein the first entrance face has an opening communicating with the central void.

10. The contact lens according to claim 1, wherein:
the exit face is delimited by a circular contour delimiting the exit face of a base of the lens,
the second face is delimited by a contour, and
the image of the virtual projection of the contour of the exit face, along the axis of symmetry, is contained in the interior of the contour of the second face.

11. The contact lens according to claim 10, wherein the image of the virtual projection of the contour of the exit face is a circle inscribed in the contour of the second face.

12. The contact lens according to claim 11, wherein the contour of the second face has a polygonal shape.

13. The contact leans according to claim 12, wherein a contour of the opening has the same geometric shape as the contour of the second entrance face.

14. The contact lens according to claim 13, wherein:
the contour of the opening has identical dimensions to the contour of the second entrance face, or
the contour of the opening has greater dimensions than the dimensions of the contour of the second entrance face so that an area defined by the contour of the opening is greater than an area defined by the second entrance face.

15. The contact lens according to claim 1, wherein the lens is one-piece and consists of a transparent plastic material.

16. A method for obtaining a contact lens for the examination and/or treatment of an eye under an incident light beam, the method comprising providing a main element comprising:
a planar first entrance face, disposed perpendicular to the beam, at least one reflective face,
a spherical exit face having a center of curvature and an axis of symmetry that is perpendicular to the first entrance face and passing through the center of curvature, the exit face configured to be applied to the eye and to focus the beam on a working point in an interior of the eye,
a second entrance face disposed facing the spherical exit face, along the axis of symmetry, and
a central void extending from the planar first entrance face until the second entrance face.

17. The method according to claim 16, wherein the contact lens is obtained by injecting a plastic material into an interior of a mold.

18. The contact lens according to claim 3, wherein the lens further includes a plurality of distinct reflective faces and a corresponding number of distinct prisms.

19. The contact lens according to claim 18, wherein the lens further includes connecting faces defining a circumference of the lens, which are interleaved between the prisms so as to connect the prisms together.

20. The contact lens according to claim 7, wherein the lens further includes a homogeneous and constant thickness defined between the internal lateral wall and the connecting face that faces it.
